(19) United States
(12) Patent Application Publication
(10) Pub. No.: US 2009/0092727 A1
(43) Pub. Date: Apr. 9, 2009

(54) WATER-DISPERSIBLE PHYTOSTEROL-SURFACTANT CONGLOMERATE PARTICLES

(76) Inventor: Daniel Perlman, Arlington, MA (US)

Correspondence Address: WESLEY B. AMES, 7031 LOS VIENTOS SERENOS, ESCONDIDO, CA, 92029, US

(21) Appl. No.: 11/867,682

(22) Filed: Oct. 4, 2007

(51) Int. Cl. A23L 1/29; A61K 9/14; A61K 31/56; A61P 3/06; A23L 1/236; A23L 2/52

(52) U.S. Cl. 426/548; 424/489; 514/169; 426/648; 426/590

(57) ABSTRACT

A hot and cold water-dispersible dry-milled composition including phytosterol-surfactant conglomerate (PSC) particles which include a blend of dry microparticulate non-ester phytosterols and a dry binary surfactant. The composition further optionally includes one or more additional dry ingredients such as anti-caking agents, anti-foam agents, natural and artificial sweeteners, non-dairy creamers and flavoring agents.

### RELATED APPLICATIONS

NOT APPLICABLE.

### FIELD OF THE INVENTION

The present invention relates to a composition and method in which phytosterols that normally do not mix with water are converted into hot and cold water-dispersible powders or granules that are useful as either food additives or dietary supplement compositions. The invention allows food and beverage manufacturers as well as consumers to achieve rapid dispersion of phytosterol powders in water-containing foods and beverages such as yoghurts, soups, sauces, coffee, juices, milk, milk-containing breakfast cereals, and the like.

### BACKGROUND OF THE INVENTION

The following discussion is provided solely to assist the understanding of the reader, and does not constitute an admission that any of the information discussed or references cited constitute prior art to the present invention.

This invention concerns particles of plant sterols, i.e., phytosterols that do not normally disperse in water without some chemical modification. With appropriate modification, phytosterol particles can be dispersed in foods and beverages, and when mixed with cholesterol in the gastrointestinal tract, can help reduce the LDL cholesterol level in the bloodstream.

Previous inventors have gone to considerable lengths to formulate aqueous suspensions from water-insoluble phytosterols, and to create novel phytosterol particle chemistries in which either the outside surface, or the entire composition of the dry particles has been chemically modified to allow dispersal of the particles in water. These phytosterol particle modifications have generally involved relatively costly liquid processing steps, e.g., solution or suspension processing, spray-drying, melt-processing, and the like.

Over thirty years ago, Thakkar et al. in U.S. Pat. No. 3,881,005 stated the following: “In order for sitosterols to be effective in lowering serum cholesterol, the medicament must reach the gastrointestinal tract in a finely divided dispersed state. Because of the hydrophobic character of sitosterols, it has not been possible to prepare a conventional tablet or capsule which will allow the thorough dispersion of the medicament in the G.I. tract. In addition, the wax-like hydrophobic surface of sitosterols makes the dispersion of the active agent in water a most difficult task. Providing a packet of finely ground sitosterols to be dispersed in water immediately before administration has not been heretofore been feasible.”

Thakkar et al. describe a complicated preparation of a pharmaceutical water-dispersible sitosterol powder. Their powder is prepared using sitosterols, an excipient or combination of excipients (such as starch, starch hydrolysate, and fumed silicon dioxide), a non-ionic or anionic surfactant (such as polyoxyethylene (20) sorbitan monostearate or sodium lauryl sulfate) and water. The surfactant is dispersed in water, the excipients are added to the surfactant-containing water, the sitosterols are added, the mixture is homogenized, deaerated, pasteurized, and the mixture is spray-dried. The resulting sitosterols that have been coated in an aqueous medium with excipient materials and surfactant are then dried, and are water-dispersible.

Ong in U.S. Pat. No. 4,195,084 describes an aqueous pharmaceutical suspension of sitosterols that includes finely divided tall oil sitosterols, a chelating agent to prevent oxidation of sitosterols, sodium carboxymethylcellulose, sorbitol, a surfactant (such as polyoxyethylene (20) sorbitan monostearate or sodium lauryl sulfate), simethicone and water. A rather complex series of steps is utilized in combining these various ingredients to produce the pharmaceutical suspension.

In more recent years, researchers at McNeil-PPC, Inc. have authored a series of patents describing water-dispersible sterols. For example, Burruano et al. in U.S. Pat. Nos. 6,054,144 and 6,110,502 describe a method for preparing sterols in a stable powder matrix that is self-emulsifying upon addition to food. A water-dispersible β-sitosterol or oryzanol powder is produced by forming a suspension of either of these sterols in an aqueous mixture of both a monofunctional surfactant (hydrophobic) and a polyfunctional surfactant (hydrophilic), and subsequently drying, e.g., spray-drying, the suspension to produce a water-dispersible powder form of the sterol. Tween 40 [polyoxyethylene (20) sorbitan monopalmitate, a liquid] is the preferred monofunctional surfactant (defined as bonding to the sterol), and is used in an approximate 1:1 ratio with Span 80 (sorbitan monooleate, a solid), the preferred polyfunctional surfactant (defined as bonding to the sterol and the other surfactant).

In U.S. Pat. No. 6,242,001 Bruce et al. describe a solid composition that is free of water, that includes a sterol/stanol or an ester thereof, and any of a variety of hydrocarbon materials that are combined, e.g., by melting them with the sterol/stanol solid to disrupt the crystallinity and improve the water-dispersibility of the material following a grinding step to form small dispersible particles. The invention of Bruce et al. claims to minimize the incorporation of surfactants and dispersants and, by adding the hydrocarbons into the solid structure of the sterol/stanol, avoids formation of the aqueous particle suspension of Burruano et al., that requires an expensive drying step, e.g., spray-drying, before obtaining a dispersible powder.

In U.S. Pat. No. 6,267,963 Bruce et al. describe a co-crystallized plant sterol-emulsifier complex consisting of plant sterol, emulsifier, and optional triglyceride oil in which the ingredients may be melted together and then co-crystallized.

In U.S. Pat. No. 6,387,411 Bruce et al. describe further solid plant sterol/stanol composition in which the sterol/stanol is combined with certain hydrocarbon compounds in the absence of water.

In U.S. Pat. No. 6,623,780 Stevens et al. describe a composition that includes one part sterol, 1.14-1.5 parts monoglyceride and 0.04-0.20 parts polysorbate (e.g., Tween 60, polyoxyethylene (20) sorbitan monostearate), that are melted together and spray-microprilled to form a powder in which 90% of the particles in water are smaller than one micron. The invention also describes heating a suspension of these particles in an aqueous food or beverage to above the melting temperature of the particles, and shearing the mixture.

In U.S. Pat. No. 6,063,776 Ostlund describes a water-soluble powder that includes an aqueous homogeneous micellar mix of plant sterol and salt of lactic acid coupled to a fatty acid, such as sodium stearoyl lactylate, in which the mixture has been water-emulsified and dried to a soluble powder.

In U.S. Pat. No. 6,677,327 Gottemoller describes an edible composition that includes plant sterols/stanols, a water-soluble or dispersible protein such as whey, soy, gluten or caseinate protein, and also lecithin, in which the composition is free of oil and has been dried to a water dispersible powder.

### SUMMARY OF THE INVENTION

Applicant has discovered a method for rendering “free” (non-esterified) phytosterol particles water-dispersible that is simpler and less costly than methods described in the prior art, because the method avoids any liquid processing step and involves only blending and dry milling of powdered ingredients. Surprisingly, instead of simply producing a mixture of separate phytosterol particles and surfactant particles, blending and dry milling of phytosterol particles with binary surfactant particles under high shear conditions produces conglomerate particles in which phytosterol particles are intimately associated with surfactant particles. In many cases, the surfactant particles are reduced in size before or during the formation of the combination particles, e.g., using high shear conditions such as those produced with a hammer mill.

Thus, a first aspect of the invention concerns a water-dispersible dry-milled composition. Typically the dry-milled composition includes phytosterol-surfactant conglomerate (PSC) particles. The conglomerate particles include a blend of microparticulate phytosterols (advantageously non-esterified phytosterols) and a binary surfactant that includes at least one non-ionic surfactant and at least one ionic surfactant, and optionally further including one or more additional dry ingredients such as anti-caking agents, anti-foam agents, natural and artificial sweeteners and flavoring agents.

In advantageous embodiments, at least 50, 75, 80, 90, or 95 percent of the phytosterols in the composition are incorporated in PSCs.

In particular embodiments, the composition and/or the phytosterol-surfactant conglomerate particles contain a blend of 1 part by weight of dry microparticulate non-ester phytosterols and from 0.1 to 4 parts by weight of a dry binary surfactant, more often from 0.2 to 2 parts by weight of a dry binary surfactant (i.e., 1 to 0.2-2), e.g., 1 to 0.2-0.5, 1 to 0.5-1.0, 1 to 1.0-1.5, 1 to 1.5-2, 1 to 0.1-1, 1 to 0.1-2, 1 to 1-4, 1 to 2-4, or 1 to 0.3-1.5.

The binary surfactant includes at least one non-ionic surfactant and at least one ionic surfactant, preferably combined in a weight ratio of from 0.3:1 to 3:1 respectively, e.g. from 0.3:1 to 0.5:1, from 0.5:1 to 1:1, from 1.5:1 to 2:1, from 2:1 to 2.5:1, from 2.5:1 to 3:1, or from 1:1 to 2:1. Likewise, in certain embodiments, an ionic surfactant is selected from the group consisting of anionic surfactants, cationic surfactants and zwitterionic surfactants; the non-ionic surfactant is selected from the group consisting of monoglycerides and combinations of mono- and diglycerides; the binary surfactant includes at least one non-ionic surfactant and at least one ionic surfactant combined in a weight ratio of from 0.5:1 and 2:1 respectively, e.g., 0.5:1 to 1:1, from 1:1 to 1.5:1, from 1.5:1 to 2:1; the binary surfactant includes mono- and diglycerides of stearic acid and/or sodium stearoyl lactylate, e.g., combined in a weight ratio of between 0.3:1 and 3:1 or other ratio as specified above for the non-ionic and ionic components of binary surfactants; the binary surfactant is a melt-sprayed mixture of at least two surfactants (e.g., a non-ionic and an anionic surfactant), e.g., in a ratio as specified above for non-ionic and ionic surfactant components of a binary surfactant.

In preferred embodiments, the phytosterols include non-esterified phytosterols and/or non-esterified phytostanols; some or all of the phytosterols are purified from vegetable oil or tall oil or both.

In certain embodiments, the average diameter of the phytosterol microparticles (e.g., non-ester phytosterol microparticles) used in the composition (generally incorporated in the conglomerate particles) is less than 25, 20, 15, 12, 10, or 8 microns or is in a range of 1-25, 1-20, 1-15, 1-10, 3-25, 3-20, 3-15, 3-10, 5-25, 5-20, 5-15, or 5-10 microns; the average diameter of the surfactant particles in the composition (generally incorporated in the conglomerate particle) is less than 150, 125, 100, 75, 50, or 25 microns, or is in a range of 10-100, 10-50, 10-25, 25-50, 50-75, 75-100, 100-150, 25-100, or 50-100 microns; the average diameter of the conglomerate particles is 10-200 microns, e.g., 10-25, 25-50, 50-75, 75-100, 100-150, 150-200, 10-50, 50-100, 100-200 microns.

In particular embodiments, the solid form of the composition is selected from the group consisting of powders, granules and tablets that are capable of rapidly disintegrating and dispersing in water, e.g., within seconds rather than minutes.

In certain preferred embodiments, a premeasured amount of the composition is provided in the form of a powder or granules, and is packaged in a single serving packet, e.g., such as is typically formed from a heat-sealable “poly-paper” film, and is preferably resistant to ambient moisture, e.g., being fabricated from multiple layers that commonly include paper and thermoplastic film, e.g., polyethylene, and optionally metal foil, e.g., aluminum, foil. In related embodiments, the premeasured amount of the composition in single serving packets (aka single use packets) ranges from 0.5 g to 2 g per packet. Such single serving packets may be further packaged in groups or sets to provide a plurality of such packets together, e.g., 2-1000, 2-100, 2-20, 5-100, 5-50, 10-100 packets.

In further embodiments, the composition also includes a suitable amount of sweetener agent, e.g., in a single serving packet this can be an amount suitable for sweetening a single serving of beverage. This sweetener may, for example, be a natural sugar or a synthetic non- or low caloric sweetener such as saccharin, aspartame, sucralose or stevia, or a combination thereof.

Likewise, instead of or in addition to a sweetener, in some embodiments the composition further includes a suitable amount of non-dairy creamer, e.g., in a single serving packet, which can, for example, be an amount suitable for a single serving of beverage (such as coffee). This ingredient can enhance the use of the composition in conjunction with coffee, hot chocolate, and any other beverage or food to which cream or non-dairy creamer lacking phytosterols might otherwise be added.

In other embodiments, a premeasured amount of the composition can be packaged in a multi-use container or bulk container.

In particular embodiments, the composition is packaged either in single use amounts (e.g., single serving powder packets) or in multi-use amounts (e.g., jars) and is labeled to be compliant with FDA regulations governing consumer use of phytosterols as either a food additive or a dietary supplement.

A related aspect of the invention concerns a dry, water-dispersible phytosterol composition which results from the process of dry milling a combination of dry particulate surfactant and a dry particulate phytosterol under high shear conditions.

Typically the composition consists essentially of conglomerate particles which are or include surfactant particles with associated phytosterol particles; the surfactant is a binary surfactant; the composition is dispersible in hot and cold water; the components, component ratios, component particle sizes, and/or resulting conglomerate particle size and/or composition are as described for the preceding aspect or otherwise described herein.

Another related aspect concerns a dry, water dispersible phytosterol composition which includes conglomerate particles which are or include irregular binary surfactant particles associated together with phytosterol particles.

In particular embodiments, at least a substantial fraction, most, or substantially all (i.e., each) conglomerate particle comprises at least one surfactant particle and at least one phytosterol particle (e.g., a plurality of phytosterol particles, such as an average of at least 2, 3, 4, 5, 7, 10, 50, 100, 200, 400, 600, 800, or 1000 phytosterol particles per surfactant particle, or is in a range of 2-5, 2-10, 2-20, 10-50, 20-100, 50-100, 50-200, 100-200, 100-400, 200-600, 400-800, or 400-1000 phytosterol particles per surfactant particle); the surfactant particles are substantially larger than the phytosterol particles; the surfactant particles have average diameters in the range of 10-150, 10-100, 25-150, 25-100, 50-150, 50-100, 50-75 microns; the conglomerate particles have average diameters in the range of 10-200, 10-100, 10-50, 25-200, 25-100, 25-75, 25-50, 50-200, 50-100, or 50-75 microns.

Likewise in particular embodiments, at least a significant fraction, most, or substantially all of the surfactant particles in the conglomerate particles are mechanically broken from larger surfactant particles.

In particular embodiments, the components, component ratios, component particle sizes, and/or resulting conglomerate particle size and/or composition are as described for the first aspect above; the dry milling is performed as described for the following aspect or otherwise described herein for the present invention.

Another related aspect concerns a method for making a water dispersible phytosterol composition by blending and dry milling a mixture of dry particulate phytosterols and dry particulate binary surfactant under high shear conditions, e.g., using a hammer mill or classifier mill.

In particular embodiments, the blending and dry milling includes milling two or more dry ingredients together (e.g., surfactant and phytosterol particles); the blending and dry milling includes milling one or more dry ingredients separately (e.g., surfactant and/or phytosterol particles) and then blending a plurality of particles together; the blending and dry milling includes blending a plurality of ingredients (e.g., surfactant and phytosterol particles) into a blend and then milling the blend.

In preferred embodiments, the composition is hot and cold water dispersible; process produces phytosterol-surfactant conglomerate (PSC) particles.

In particular embodiments, the conglomerate particle size and/or composition are as described for the first aspect above or otherwise described herein for the present conglomerate particles.

The present invention also provides methods for using the present phytosterol-surfactant conglomerate particles and corresponding composition. Such methods include use of such particles and compositions in the diet of individuals and/or in preparing foods or beverages by incorporating the conglomerate particles or corresponding compositions in an edible food or beverage. Thus, for example, another aspect concerns a method for supplementing the diet of an individual with phytosterols by adding an amount of a dry, water dispersible composition comprising phytosterol-surfactant conglomerate (PSC) particles to a food or beverage item (usually a water-containing food or beverage item), and can further include an individual ingesting at least a portion of the food item or beverage.

In particular embodiments, the amount included in a single serving of the food item or beverage is an amount which does not significantly change the taste, texture, and/or mouth feel of the food item or beverage, and/or the amount is a cholesterol-lowering amount; the food item or beverage is yoghurt, soup, sauce, coffee, juice, milk, a milk-containing breakfast cereal, mashed potatoes, refried beans, pasta, or rice.

In particular embodiments of this aspect or any embodiment thereof, the composition is a composition as described for an aspect above, or otherwise described herein for the present invention.

In another aspect, the methods for using the present compositions include a method for reducing the uptake of dietary cholesterol by ingesting a cholesterol lowering amount of the present dry milled phytosterol-surfactant composition.

In particular embodiments, the dry milled phytosterol-surfactant composition is as described for an aspect above or otherwise described herein. Also in particular embodiments, the dry milled phytosterol composition is ingested as part of a food or beverage item of a type indicated herein, e.g., water, juice, coffee, milk, yoghurt, cereal and milk, sauce, soup, mashed potatoes, refried beans, pasta, rice, and other such water containing foods and beverages.

Additional embodiments will be apparent from the Detailed Description and from the claims.

### DETAILED DESCRIPTION OF THE PREFERRED EMBODIMENTS

### Definitions

The following definitions of terms are provided to assist the understanding of the reader. For terms that are not defined below, the common definition is assumed as provided in the current edition of Webster's International Dictionary or alternatively, provided in a standard organic chemistry textbook such as Organic Chemistry (5^{th} Edition) by Leroy Wade (Prentice-Hall, Inc). As used in this description and the accompanying claims, the following terms shall have the meanings indicated, unless the context requires otherwise.

In connection with the present compositions, the term “water dispersible” means that one gram of the composition will disperse in 240 grams of water at 70 degrees F. within 20 seconds (and preferably no more than 17, 15, 12, 10, 7, 5, 4, or 3 seconds) with no more than moderate manual mixing.

The phrase “hot and cold water-dispersible” refers to compositions described herein, that may be readily dispersed when placed into water at any temperature ranging from hot to cold, that is, a range of at least 0° C. to 100° C. unless otherwise specified. Of course, it is recognized that similar to the dissolution and dispersion processes for other materials, generally dispersal of the present compositions in cold water will proceed more slowly than in hot water under the same mixing conditions. Highly preferably, such dispersion behavior occurs for a range of aqueous or water-containing foods and beverages, for example, hot water, hot beverage or hot aqueous or aqueous-containing food (e.g., coffee, tea, hot chocolate, hot oatmeal (or similarly other hot cereals from wheat, rye, or rice, and the like), soup, stew, hot cereal with milk, mashed potatoes, refried beans, pasta, or rice (e.g., steamed rice)) typically at a temperature ranging from 50° C. to 100° C., or alternatively into, for example, cold water, cold aqueous beverage or cold aqueous food (e.g., orange juice, milk, yoghurt, dry cereal with milk) at a temperature ranging from 0° C. to ambient temperature, and furthermore, stirred or shaken. The term “dispersible” refers to the composition, and in particular the microparticulate phytosterols described herein, becoming essentially uniformly distributed throughout a serving or other quantity of water or aqueous food or beverage medium in which the composition is mixed.

The term “dry milled blend” or other “dry-milled” material refers to a dry material, usually a combination of two or more dry ingredients, that are premeasured or metered and fed into a mill, generally a powder mill, and milled. For a dry milled blend, the two or more ingredients are milled together, or alternatively, ingredients milled separately and thereafter combined into a blend, or alternatively, mixed into a blend before milling (using shaking, stirring, or machine blending, e.g., a ribbon blender) and then processed through a mill, e.g., a hammer mill (see below), Generally the dry milling reduces the particle size of one or more of the ingredients. The latter method is a preferred method in the present invention. Besides reducing particle size, the milling process may act to combine two or more ingredients in the blend into a composite or conglomerate material as in the present invention.

In the context of milling the present materials, the term “high shear conditions” is used in a manner consistent with milling of edible products, and in particular indicates that the milling conditions are such that significant size reduction of 250 micron dry P28 binary surfactant particles or other melt blend of sodium stearoyl lactylate and mono- and diglycerides in a 1:1 ratio will occur. Highly preferably, such size reduction will occur for any dry surfactant (e.g., binary surfactant) particles of the same or similar size used in the present compositions.

As used in connection with the present invention, the term “particulate” means the referenced material is in the form of particles, usually dry particles. The size of the particles may be of any of a range of sizes, including, for example, size ranges from fine powders to granules. In many cases, the component phytosterols will be in the range of 1-100 microns and/or the component surfactants will be in the range of 10-250 microns.

For referring to the sizes of particles in this invention, it is recognized that in many cases the particles are substantially non-spherical. Thus, for a particle, the term “diameter” refers to the diameter of a spherical particle having equivalent volume. This can be acceptably approximated by the mean linear dimension of the particle for lines passing through the center of mass of the particle, which itself may be acceptably approximated by taking the mean of the thickness of the particle along 2 orthogonal axes of a coordinate system, with one of the axes aligned with the longest dimension of the particle. Such determination may be made, for example, using a microscope with a suitable length scale. The term “average diameter” refers to the volume medium diameter D(v,0.5), meaning that approximately 50 volume % of the particles have an equivalent spherical diameter that is smaller than the average diameter and approximately 50 volume % of the particles have an equivalent spherical diameter that is greater than the average diameter.

The term “dry microparticulate non-ester phytosterols” or simply “microparticulate phytosterols” or “phytosterol microparticles” collectively refers to very small phytosterol and/or phytostanol particulate material (either separately or in combination), that is in its natural or “free” chemical state (rather than chemically modified). Free phytosterols are typically isolated and purified from nature (e.g., from vegetable oils or from tall oils). The qualifying term, “non-ester” means that the phytosterols have not been chemically modified at the hydroxyl site in the molecule by fatty acid esterification as is typically done to render the phytosterols fat-soluble.

The term “binary surfactant” as used herein means the same as “hybrid surfactant”, and is discussed elsewhere herein. Briefly, a binary surfactant includes at least one non-ionic (having no ionizing or salt-type groups), predominantly hydrophobic surfactant and also at least one ionic (having one or more ionizing group), predominantly hydrophilic surfactant. The term “surfactant” or surface-active agent as used herein, refers to an agent, usually an organic chemical compound that is at least partially amphiphilic, i.e., typically containing a hydrophobic tail group and hydrophilic polar head group. These properties typically allow solubility of the surfactant in organic solvents as well as in water, and allow the surfactant to promote solubilization or at least dispersal of fatty/waxy materials in water and water-containing foods.

In the present context, it is proposed that the binary surfactants described herein promote dispersion of hydrophobic sterol materials in water by forming micelles in which fatty acid tails can form a hydrophobic core associating with the sterol particle while their polar or ionic heads (e.g., sodium lactylate) can form an outer shell that maintains favorable contact with water and water-containing foods. As stated above, the present invention utilizes a binary surfactant, e.g., a blend of an anionic and a non-ionic surfactant in which the efficacy of these surfactants has been enhanced by their pre-combining at the molecular level. Applicant considers the binary surfactant particularly effective in dispersing the inherently waxy phytosterol particles in a water-based beverage or food medium. The fact that more complicated and expensive methods including solution and melt-processing of sterol particles have been considered necessary for achieving such water dispersal, suggests that the present method requiring only dry-blending of ingredients has hitherto escaped discovery.

Indication herein that surfactant particles and phytosterol particles are “associated” or “associated together” means that the particles interact is such manner that the respective particles “stick” together in a dry state. Without being limited to any particular type of interaction, the association may result from energetically favorable interactions such a charge:charge, charge:polar, and/or polar:polar interactions. For example, in the present ionic-nonionic particle mixtures, an energetically favored reduction in electrostatic self-repulsion interaction energy between the ionic surfactant particles may be achieved by dilution and association with the non-ionic phytosterol particles.

The reference to one or more “additional dry ingredients” in the composition pertains to optional agents added to the principal phytosterol and surfactant ingredients. These include anti-caking agents, anti-foam agents, natural and artificial sweeteners and flavoring agents, in which all of these agents having their conventional meanings.

The term “monoglyceride” refers to any of the fatty-acid glycerol esters where only one fatty acid group is attached to the glycerol group. Mono- and diglycerides are non-ionic surfactants consisting of a mixture of monoglycerides and diglycerides in which one and two fatty acid groups are attached to the glycerol group; examples are glycerol mono- and distearate and glycerol monolaurate or monopalmitate.

The term “anionic surfactant” refers to a surfactant in which the principal functional group (the “head” of the molecule) is negatively charged, such as stearoyl lactylate (−) with a positively charged sodium counter-ion (a preferred surfactant herein). In general commercial use, anionic surfactants are typically used in laundering, dishwashing and shampoos. The efficacy of these surfactants is generally reduced by the positively charged ions in hard water (calcium and magnesium). Commonly used anionic surfactants include the alkyl sulphates, alkyl ethoxylate sulphates and soaps.

The term “non-ionic surfactant” refers to a surfactant in which the principal functional group is not ionized, i.e., it carries no electrical charge, and in the context of the present invention, is a lipophilic surfactant that exhibits a strong chemical association with phytosterols. Besides the mono- and diglycerides being used herein and used elsewhere in foods, non-ionic surfactants include various ethers of fatty alcohols. In general commerce (laundry products, household cleaners), non-ionic surfactants can be beneficially combined with anionic surfactants because the efficacy of the non-ionics is not compromised by the positively charged ions in hard water.

The term “zwitterionic” or “amphoteric” as used herein refers to surfactants that may carry both a positive and negative charge depending on the pH of the medium. Typically, they may be combined with the other classes of surfactants. Whereas the positive charge is almost always ammonium, the source of the negative charge may vary (carboxylate, sulphate, sulphonate). These surfactants are frequently used in shampoos, other cosmetic products, and also in hand dishwashing liquids because of their high foaming properties, e.g., alkyl betaine

The term “phytosterol-surfactant conglomerate” particles (abbreviated PSC particles) is defined and described elsewhere herein. The PSC structure and its method of manufacture are novel because as described herein, it is a physical conglomerate of (i) non-ester phytosterol microparticles and (ii) larger particles of binary surfactant (e.g., an anionic surfactant plus a non-ionic surfactant) that are mutually adherent. The conglomerate particles are produced by dry milling of these blended ingredients in appropriate proportions.

The term “natural and artificial sweeteners” refers to sugars and other naturally occurring sweeteners (natural sweeteners), and also synthetic sweeteners or sugar substitutes such as aspartame, sucralose, saccharin, stevia and the like (artificial sweeteners.

The term “anti-caking agent” refers to an edible inert material that can be added to the composition described herein to promote the hot and cold water-dispersibility of the composition. For example, amorphous hydrophilic silicon dioxide such as Cab-O—Sil® M5 or Flo-Gard® AB (described elsewhere herein) may be added at levels up to 2% by weight of the composition to remain within the limits prescribed by the U.S. FDA for use as an anti-caking agent direct food additive.

For the present compositions, a “cholesterol lowering amount” is an amount which significantly reduces the uptake of co-ingested cholesterol for an individual with normal cholesterol uptake. The U.S. FDA presently specifies that an individual should consume at least 400 mg non-ester phytosterols per serving of a cholesterol-lowering food at least twice per day to achieve a meaningful health benefit. For a composition of the present invention that contains 50% by weight phytosterols, 800 mg of the composition would certainly be considered a “cholesterol-lowering amount.” However, for compositions that contain a smaller proportion of surfactant to phytosterol, and for individuals who combine the present composition with other cholesterol-lowering agents, the cholesterol-lowering amount would typically be smaller, e.g., 2-fold or 4-fold smaller.

### General Description

This invention relates to the use of dry powder milling to produce heterogeneous conglomerate particles that contain both phytosterols, usually non-ester phytosterol microparticles, and surfactant materials. The term “non-ester phytosterols” (aka “free phytosterols”) includes phytosterols, phytostanols and combinations thereof. The invention also relates to the compositions of dry blends used to produce these conglomerate particles. The invention also provides dry-blending methods that cause structural changes in the starting materials, e.g., fracturing of large surfactant particles, and new interactions between the fractured surfactant and the microparticles of phytosterols, which are usually much smaller. These interactions result in the formation of heterogeneous conglomerate particles whose constituents easily disperse in both hot and cold water as well as aqueous foods and beverages. From visual inspection (light microscopy) of the anisotropic distribution of ingredients within the irregular-appearing conglomerate particles of the present invention, it is apparent that these conglomerates differ in essential ways from prior art composite particles, such as those produced by solution-coating, melt-blending and/or spray-drying.

The present particles can be used in many different types of aqueous solutions and/or suspensions, as well as in a large variety of food which are prepared using such aqueous solutions and suspensions. For example, these particles may be used in liquids such as water (e.g., plain, flavored, or fortified), fruit and/or vegetable juices and juice blends (e.g., orange, apple, cranberry, grape, raspberry, blueberry, and carrot juices as well as other fruit and/or vegetable juices and juice blends) steeped or brewed beverages (such as coffee, tea, and herbal teas), milk, dairy products containing significant amounts of water (e.g., yoghurt, cottage cheese, cheese), apple sauce, canned fruits, foods which are cooked using water or other aqueous liquid as an ingredient (e.g., soups, stews, mashed potatoes, refried beans, pasta, rice, and the like, or can be added to foods which contain significant amounts of water (e.g., raw eggs, which can then be used in essentially any manner for which raw eggs are suitable).

Without being bound or limited by theory, it is hypothesized that phytosterol microparticle dispersal from the conglomerates described herein, occurs as water (or other aqueous material) contacts and rapidly dissolves the surfactant material in the conglomerate particles, whereupon the high local concentration of solubilized surfactant wets the surface of the many (e.g., tens to hundreds) associated phytosterol microparticles within each conglomerate particle. Surfactant remaining on the surface of the freed phytosterol microparticles allows the microparticles to freely mix with the surrounding aqueous medium.

The water-dispersible heterogeneous conglomerate particles, that contain phytosterol microparticles plus larger fractured pieces of surfactant, are generally free-flowing dry powder particles that more specifically include: (i) phytosterol microparticles whose weight average diameter is usually ≦25 microns, and preferably ≦10 microns in diameter), and (ii) a combination of at least one hydrophobic surfactant, e.g. a non-ionic mono- and diglyceride, and at least one hydrophilic surfactant, e.g., an anionic surfactant. The hydrophobic and hydrophilic surfactant components may be conveniently purchased as a binary hybrid mixture co-mingled at the molecular level in which the dual surfactant exhibits the beneficial properties of both surfactants. This hybrid or “binary surfactant” blend is, in turn, conveniently dry-blended with microparticulate phytosterols under high shear conditions to form a water-dispersible powder.

For the purposes of defining the scope of the present invention, it is clearly possible that a single surfactant molecule may be found in which the hydrophobic and hydrophilic surfactant properties required herein, have been merged. Optional excipients such as hydrophilic amorphous silica (silicon dioxide) may also be added, e.g., as anticaking or flow agents to prevent clumping or caking of the powder during storage, particularly if the powder is exposed to humidity. An optional antifoam agent may also be added to facilitate dispersion by combating foam formation. Other food grade ingredients such as natural and/or artificial sweeteners and/or non-dairy creamers may also be combined with the water-dispersible phytosterols.

Applicant finds no prior art example of a phytosterol (specifically including a non-ester phytosterol) composition that is water-dispersible, in which the composition is produced by dry-milling of ingredients without the need to subject the phytosterols to either a melt, a solution or a suspension-processing step. Such liquid or melt-processing steps require heating, cooling, and subjecting the phytosterols to a series of controlled processing steps that are costly. In contrast, the present invention involves only dry-blending/milling of selected ingredients, one of which is a phytosterol preparation, usually a non-esterified phytosterol powder. As demonstrated in the present invention, water-dispersible phytosterol microparticles can be produced using mechanical milling of dry commercially available ingredients. This milling is simple and cost-effective, and obviates the need for using more complicated and generally more expensive processing methods.

The present phytosterol-surfactant compositions can be used in a similar manner to other phytosterol compositions, especially other water soluble or dispersible phytosterol compositions. One of the major uses for such compositions is to reduce the uptake of dietary cholesterol, e.g., by co-ingestion (usually in the same meal or even in the same food item) of the phytosterol with cholesterol-containing food items. Such uses are described in patents cited in the Background, each of which is incorporated herein by reference in its entirety. The present compositions can be used in the same or similar manner to the dry phytosterol compositions described in those patents. Description of such uses will therefore not be repeated herein.

### Combining Surfactant and Phytosterol.

Two prior art methods (described above in the Background) for uniting a surfactant with phytosterols to form a dry preparation that is water-dispersible, include (i) suspending and coating phytosterol particles in an aqueous surfactant medium and then spray-drying the suspension, and (ii) melt-blending a surfactant and phytosterols, and then spraying and cooling the mixture to form microparticles. Unlike these prior art methods that involve liquids and melts, the present invention utilizes only dry materials.

At the outset of planning experiments relating to the present invention, Applicant sought a simpler and less costly method for producing water-dispersible non-ester phytosterol powders. Accordingly, Applicant obtained samples of powdered microparticulate non-ester phytosterols from ADM (CardioAid® M, Decatur Ill.) and from Cognis Nutrition and Health (Vegapure® FS, La Grange, Ill.). The diameter of the majority of particles in these phytosterol preparations is ≦10 microns. In addition, three surfactant materials that are suitable for dispersing fatty materials were obtained, i.e., a non-ionic hydrophobic, an anionic, and a mixed surfactant produced by Kerry Bio-Science, Inc. (Rochester, Minn.). These surfactants included:
(a) mono- and diglycerides of stearic acid (Myverol® 18-04 K),
(b) sodium stearoyl lactylate (Admul® SSL 1078 K) and
(c) a binary surfactant as described above (Myvatex® P28 XLK) consisting of a combination of surfactants (a) and (b). The Myvatex P28 binary surfactant is described as containing between 50 and 75% by weight sodium stearoyl lactylate and 25-50% by weight mono and diglycerides of stearic acid. With visualization by light microscopy the product consists of smooth spherical microbeads ranging from approximately 150 to 250 microns in diameter. The material is produced by molecular comingling of (a) and (b), such as by co-spraying a melt blend.

The first small scale dry powder blending experiments described herein were carried out using a horizontally rotating single blade coffee grinder (Braun AG model KSM11). Dry powdered non-ester phytosterol (49% by weight of Cognis Vegapure® FS) was mixed together with an equal proportion (49%) of surfactant A [mono- and diglycerides of stearic acid (Myverol® 18-04 K)], and 2% by weight of hydrophilic microparticulate silica (PPG Flo-Gard AB) as an anticaking agent. Similar blends were made replacing surfactant A, first with surfactant B [sodium stearoyl lactylate (Admul® SSL 1078 K)] and then with surfactant C [the binary surfactant described above (Myvatex® P28 XLK)]. Each of the resulting mixtures was milled twice in the coffee grinder (15 seconds each time with shaking to reduce powder settling). One gram quantities of each of the resulting milled powders were added and stirred into 240 g (8 oz) of hot water (80° C.) and cold water (4° C.). Only surfactant (C) provided satisfactory dispersal of the phytosterols in both hot and cold water.

Thus, the above-described binary surfactant and a small amount of anti-caking agent can be combined with microparticulate non-ester phytosterols and subjected to the impact and shear forces of dry powder milling to produce a new powder that rapidly disperses in water. Both Cognis Vegapure® FS phytosterols (90% of particles≦10 microns in diameter) and a similar product, ADM CardioAid® M phytosterols produced satisfactory results. Once dispersed in a glass of water or a beverage, the microparticulate phytosterols remained in suspension for many hours without settling. After 1-2 days without agitation, the phytosterol particles that had settled from the suspension were easily and conveniently re-suspended by stirring or shaking the liquid. The desirable stability of these phytosterol suspensions, i.e., their slow settling rate, is in part attributable to the effectiveness of the surfactant, but in large measure is due to the small particle size of the phytosterol microparticulate ingredient used in the powder recipe, and the ability of the microparticles to remain discrete while in suspension.

In fact, after dispersal in water, the microparticles do not show any clumping when viewed under a microscope, i.e., they retain their original size, i.e., 90% of particles ≦10 microns in diameter. This is important because it helps assure that upon ingestion, these microparticulate phytosterols are well dispersed in aqueous foods, and have maximum surface area and ability to be emulsified in vivo during digestion. These properties also help assure that the phytosterols will have maximum bioavailability in the gastrointestinal tract to compete with, and reduce the absorption of cholesterol into the bloodstream. Again, this discussion is meant to emphasize the advantage of choosing and utilizing phytosterol powders in the recipes described herein with as small a particle size as possible.

It was anticipated that the high impact/high shear milling of the coffee grinder described above would substantially reduce the mean particle size of the ingredients. A phase contrast microscopic examination and comparison of the milled product with the input ingredients at 150× and 600× magnification was revealing. The Myvatex P28 binary surfactant ingredient is added as large round smooth microbeads (˜150-250 microns diameter) that appear black under phase contrast. These microbeads are cleaved during milling to much smaller irregularly shaped particles. Surprisingly, most of the phytosterol microparticles that had appeared ubiquitous and bright under phase contrast before blending (less than 10 microns diameter) simply disappeared from view after blending. In fact, it became clear that the phytosterol particles had been captured within the P28 surfactant material during milling.

Irregularly shaped mixed conglomerate particles measuring approximately 50-75 microns in diameter were formed in place of the original material. That is, the ≦10 micron phytosterol particles were co-mingled with the fragmenting surfactant material to form particles that were much larger than the original phytosterol particles. These new composite particles, herein termed phytosterol-surfactant conglomerates (abbreviated PSCs herein), aka “phytosterol-surfactant conglomerate particles”, are fully water-dispersible. The term “conglomerate” is used because the phytosterols appear to have become imbedded in the surfactant much like the stones in an asphalt road material. Microscopic inspection of the 50-75 μm diameter PSCs suggests that the surfactant forms a substantially continuous external phase in which the much smaller phytosterol particles are embedded.

Microscopic examination confirmed that the milling process does not cause any significant amount of segregated clumping of either surfactant alone or phytosterols alone. That is, essentially all of the larger particles are PSCs rather than homogenous clumps of surfactant or phytosterol material. Definitive characterization of the composite, or conglomerate, nature of the PSC particles was obtained by viewing these particles with a phase contrast microscope at 150× and 600× magnification. Small volumes (2 to 5 μl microdroplets) of distilled water were moved into contact with PSC particles that were held and viewed under a glass coverslip positioned on a glass hemocytometer slide on the microscope stage. As microdroplets of water migrated (by capillary flow) into contact with PSC particles, the irregularly shaped, phase-dark P28 surfactant material in the PSC particles was observed to rapidly if not instantly dissolve, releasing the small bright-phase phytosterol microparticles that had been held within the particles.

While the small amount (2% by weight) of silica anti-caking agent could not be microscopically visualized, it was apparent that the silica was helpful in maintaining the newly forming PSCs as free-flowing non-clumping particles. The overall process renders the phytosterols dispersible in either hot or cold water, as well as aqueous beverages, water-containing foods and the like.

After successfully producing small amounts of water-dispersible phytosterol powders utilizing a coffee grinder as described above, Applicant proceeded to scale up the process utilizing the same dry ingredients described above, but milled using high capacity commercial powder milling machinery.

According to the methods described herein, the commercially available dry powdered phytosterols described above (edible and typically having a purity of greater than 95%, and typically 98% by weight, refined from either vegetable oils or tall oils) are first preferably mechanically mixed to “approximate homogeneity” with the above-described binary surfactant before being subjected to high impact blending with sufficient shearing force to cause surfactant particle size reduction while simultaneously forcing the binary surfactant agent together with the much smaller particles of phytosterols to form the phytosterol-surfactant conglomerates (PSCs). Any one of several mechanical methods, such as low shear ribbon blending, is used to achieve initial ingredient mixing, followed by high shear milling to achieve particle size reduction and co-mingling of the surfactant and phytosterol powders. For example, hammer mills or classifier mills such as a FCM 350 classifier mill manufactured by the Fitzpatrick Company (Elmhurst, Ill.) will effectively handle these materials.

The uniqueness of this process is that it is much simpler and more cost-effective than using the prior art solution, melt and/or spray-drying processes to unite phytosterols with surfactants.

To summarize, as already stated, for the purpose of biological efficacy (bioavailability), and so that water-dispersed phytosterol material will remain in aqueous suspension in beverages and food as long as possible, the phytosterols are preferably provided in as fine a microparticulate material as possible. These phytosterols are combined as a dry powder (commonly ≦10 microns) with a surfactant, advantageously a commercially available dry-mixed surfactant in which the surfactant includes at least one hydrophobic or non-ionic surfactant component, e.g., a monoglyceride, and at least one hydrophilic or ionic surfactant component, e.g., sodium stearoyl lactylate. These surfactant components can be purchased as a pre-mixed co-mingled mixture. Optional excipients may also be added to the dry blend to facilitate later aqueous dispersal of the phytosterols.

The dry phytosterol microparticulate powder and surfactants can be milled using, for example, high shear blade or pin-disc blending, or hammer milling of the dry powder blend without applying excessive compressive forces or excessive heating that could cause clumping. Powder blends containing an equal weight proportion of microparticulate phytosterols and binary surfactant (or even a combination of 70-80 parts by weight phytosterols and only 20-30 parts surfactant) together with an anti-caking agent, such as 2 parts hydrophilic amorphous silica, can be dry-milled together and rendered water-dispersible. The resulting PSC particles containing sterol microparticles and binary surfactant (with optional excipients) may be dispersed in aqueous beverages and foods by stirring or shaking the powder with either hot or cold aqueous beverage or even a water-containing food.

One of the important advantages of the present invention over the prior art methods for producing water-dispersible phytosterols is the simplicity of the manufacturing method, resulting in a more cost-effective final product. Typical water-dispersible non-esterified phytosterols are currently being sold in the marketplace at approximately two to three times the price of regular phytosterols (for the same quantity of active phytosterol material). For example the Cognis Corporation currently sells regular non-esterified phytosterol powder (98% actives) in bulk quantities for approximately $15 per kg. Cognis sells the same material in a water-dispersible form that contains only 40% by weight active sterols for approximately the same price per kg. In other words, the phytosterol component of the water-dispersible material is 2.5 times more expensive in the water-dispersible form. By contrast, the materials and manufacturing method of the present invention are expected to add only modestly to the cost of the original phytosterols, e.g., an estimated 10-25% cost increase rather than the 150% price increase mentioned above.

In accordance with the description above, an example of a blend that may be dispersed in hot or cold water, microparticulate non-ester sterols, e.g., “CardioAid M” brand micronized free sterols manufactured by Archer Daniels Midland Company (Decatur, Ill.) or “Vegapure FS” brand micronized free sterols manufactured by Cognis Nutrition and Health (La Grange, Ill.) are combined with approximately an equal weight proportion of Myvatex P28 XL K surfactant produced by Kerry Bio-Science, Inc. (Rochester, Minn.). This Myvatex P28 hybrid surfactant mixture contains between 50 and 75% by weight sodium stearoyl lactylate and 25-50% by weight mono and diglycerides of stearic acid (e.g., glyceryl monostearate) that are described as having been pre-blended at the molecular level. As a further addition to the blend, an excipient consisting of approximately 2% by weight hydrophilic fumed silica, e.g., Flo-Gard AB hydrophilic silica (PPG Industries, Inc., Pittsburgh, Pa.), is added. This three ingredient dry blend is subjected to high shear blending by passage through a high speed rotating blade blender to prepare it for dispersal in an aqueous beverage or food. For preparing larger quantities of material in commercial production, a hammer mill (see below) was utilized.

It is interesting to compare the present powder milling method with the more complex and costly prior art methods for converting waxy sterol particles to water-dispersible or water-soluble particles by either forming water-borne surfactant coatings or other emulsifier combinations with the sterol particles (see above, Thakkar at al., Burruano et al. and Ostlund) or by modifying the overall chemical composition of the sterol particles, e.g., by melt-blending the sterols to make them hydrophilic (see above, Bruce et al. and Stevens et al.). In fact, it is remarkable that commercially available unmodified sterol particles described herein can be dispersed in a beverage by simply co-blending the dry sterol particles with a dry hybrid-type surfactant (e.g., Myvatex P28 described above) and a dry excipient before use. It is both convenient and desirable that all of the ingredients comply with Food and Drug Administration regulations governing direct food additives.

### Hammer Millinci Achieves Both Particle Size Reduction and Adhesion Between Phytosterol and Surfactant Particles.

One example of a preferred powder processing method that can effectively convert commercial quantities (thousands of pounds) of non-ester phytosterol powders to a water-dispersible product is illustrated as follows: Fifty percent by weight non-ester phytosterol powder (e.g., Cognis FS sterols having a small particle size, i.e., ≧90% of material being ≦10 microns in diameter) is initially mixed using non-shearing ribbon blending with approximately 48% by weight of a binary surfactant having a substantially larger particle size (e.g., Myvatex P28 powder-50-100 micron material), and 2% by weight silica anti-caking agent, e.g., PPG Flo-Gard AB. Once mixed, high shear hammer milling is a preferred means for achieving particle size reduction of the surfactant, and accompanying adhesion of the surfactant particles around groups of phytosterol particles. The silica anticaking particles appear to form a coating around surfactant-coated phytosterol particles to reduce or prevent these surfactant-coated particles from cohering.

Hammer mills and their general operation are described on the web at: www.feedmachinery.com/cilossary/hammer mill.php. The following discussion (partially extracted from this reference) is provided to help understand this milling process in the context of the present invention. Hammer milling is able to achieve particle sizes reduction, providing that the material is fragile under the impact conditions of the mill. Generally hard particles milled in hammer mills become substantially spherical, with a surface that appears polished. By contrast, the somewhat waxy surfactant and phytosterol materials being milled in the present invention are relatively soft and somewhat cohesive. The resultant milled particles appear more irregular than spherical. The hammer milling operation produces some heat, so that care should be used to avoid ingredient melting. (In the present case, the P28 surfactant component has a low melting temperature, i.e., 110-115° F.).

The principal functional components of the hammer mill include: (a) a variable speed delivery or vein feed mechanism to introduce the mixture to be ground into the mill chamber or housing, and into the path of the rotating hammers, (b) a rotor that includes a series of machined disks mounted on a central horizontal shaft, (c) free-swinging hammers that are suspended from rods running parallel to the central shaft and through the rotor disks (these hammers carry out the function of cutting and/or smashing the ingredients in order to reduce their particle size), and (d) a perforated screen/sieve with either gravity or air-assisted removal of the ground product. The screen ensures that the particles meet a specified maximum mesh size. The size, thickness, design, and placement of hammers is determined by operating parameters such as rotor speed, motor horsepower, and open area in the screen. Optimal hammer design and placement will provide maximum contact with the feed ingredient. Hammer mill rotor speeds generally range from approximately 1,800 rpm to about 3,600 rpm. Hammers should be balanced and arranged on the rods so that they do not trail one another. The distance between hammer and screen should be about 4 to 14 mm for size reduction of cereal grains. The velocity or tip speed of the hammers is critical for proper size reduction. Tip speed is the speed of the hammer at its tip or edge furthest away from the rotor, and is calculated by multiplying the rotational speed of the drive source (shaft rpm) by the circumference of the hammer tip arc. A common range of tip speeds seen in hammer mills is in the range of 16,000 to 23,000 feet per minute or 180-260 mph.

Impact is the primary force used in a hammer mill, and anything which increases the chance of a collision between a hammer and the target material is helpful. Hammers can also be selected or modified to have a relatively sharp leading edge (or conversely run with a blunt leading edge). Experiments in which the binary surfactant and the phytosterols of the present invention have been combined and co-milled suggest that sharpened hammers may produce PSC particles that disperse more easily in water than those milled using blunt end hammers. In addition to the hammers, the amount of open area and mesh size of the hammer mill sieving screen determines the final particle size and grinding efficiency. The screen is designed to maintain physical integrity and provide the greatest amount of open area. Not enough screen open area per mill horsepower results in the generation of heat. The removal of sized material from a hammer mill is a critical design feature. Proper output of material affects not only the efficiency of operation, but also particle size. When the correct ratio of screen area to horsepower is used and proper distance between hammers and screen face is maintained, most of the correctly sized particles will exit the screen in a timely manner. Most newer hammer mills are equipped with an air-assist system that draws air into the hammer mill with the product to be ground. Systems are designed to provide reduced pressure on the exit side of the screen to disrupt the fluidized bed of material on the inner face of the screen, thus allowing particles to exit through screen holes.

### Use of Increasing Proportions of Phytosterols Relative to Surfactants.

As indicated above, the Myvatex P28 anionic+non-ionic hybrid surfactant manufactured by Kerry BioScience is a potent agent for dispersing microparticulate phytosterols such as ADM's CardioAid M material. For example, a 50-50 blend of phytosterols and Myvatex P28 surfactant which is supplemented with 2% by weight Flo-Gard AB hydrophilic amorphous silica and then dry-blended using a high shear blade blender produces a powder that is easily dispersible in both hot and cold water, beverages and food, etc. Blend of as much as 70 parts (or more) by weight phytosterols to 30 parts Myvatex P28 surfactant, and 2 parts by weight hydrophilic silica can also dispersed in both hot and cold water. The results of preliminary experiments in which the weight ratio of phytosterol to Myvatex P28 surfactant to phytosterol is increased stepwise above 50:50 (e.g., to 60:40 and 70:30) indicate that dispersal of the sterols in hot and cold water readily occurs. A somewhat greater mixing time may be required for achieving full dispersal with the higher ratios of sterols to surfactant.

### Stability of Dry Powder Blend.

Dry powder blended samples described above (e.g., 50%-70% ADM microparticulate CardioAid M sterols+28%-48% Kerry BioScience Myvatex P28 surfactants+2% PPG Flo-Gard AB hydrophilic amorphous silica) that were combined and dry-milled with a blade cutter/blender have been exposed to ambient moisture conditions at room temperature for many months without showing any tendency to cake, clump or lose aqueous dispersibility. Still, it may be advisable to exclude moisture from the dry powder blends because one or more of the ingredients may eventually attract moisture. Similar blends combining 50% Cognis FS phytosterols, 48% Myvatex P28 surfactant, and 2% PPG Flo-Gard AB have been hammer milled and packaged for Applicant by Spectrum Packaging, Inc., (Springfield, Ill.). Approximately 0.85 g quantities of this milled blend have been successfully commercially packaged in single serving-sized laminated packets designated “C1S paper/polyethylene” measuring approximately 1.5″×2.5″, as well as in similar packets having an additional aluminum foil barrier layer, i.e., “C1S paper/polyethylene/foil/polyethylene.” These packets exclude moisture, while providing the consumer with a convenient pre-measured single serving quantity of the water-dispersible non-ester phytosterols. In this example, at least a 400 mg quantity of non-ester phytosterols is provided, pursuant to the current FDA guidelines for minimum recommended amount of phytosterols per serving.

### Foam Control in Liquid Systems.

Depending upon the ingredients contained in the aqueous liquid that is being used to suspend and disperse the phytosterol and surfactant powder, temporary foaming may occur to varying degrees. To reduce or eliminate foaming, an edible anti-foam agent may be incorporated into the powder blend. The anti-foam agent is selected to avoid clumping of the particulate ingredients that would interfere with aqueous dispersal of the powder. For example, a dry powder anti-foam agent such as “1920 Powdered Antifoam” manufactured by Dow Corning (Midland, Mich.) may be used. This product contains approximately 80% by weight maltodextrin combined with 20% by weight FDA-approved food grade polydimethylsiloxane.

### Product Applications.

Numerous applications exist for water-dispersible phytosterols (abbreviated herein WDPs) in the areas of foods, beverages, dietary supplements and pharmaceuticals. Formulated as a dry powder that is readily dispersible in water as described herein, WDPs can be packaged as described above in pre-measured quantities of powder, e.g., in packets, that are readily opened at the time of use, and sprinkled into foods and beverages. Alternatively, the powders may be packaged in edible capsules for ingestion as dietary supplements to reduce plasma cholesterol levels.

The powders may also be readily combined with other edible powders such as powder drink mixes, dry soup mixes, non-dairy coffee creamer and/or natural or artificial sweetener (e.g., sucralose or aspartame) or similarly packaged in appropriate amounts in single use packets. Alternatively, large quantities of the WDPs may be used in the commercial production of processed foods and beverages that require supplementation with phytosterols or for which such supplementation is desired.

### FDA Regulatory Matters.

The U.S. Food and Drug Administration regulates many surfactants as direct food additives, including the levels of use and types of foods to which those surfactants may be added. For example, certain non-ionic surfactants including the sorbitol fatty acid esters (e.g., Tweens (polysorbates) may be limited to about 0.5% by weight of typical dry food mixes. However, other non-ionic surfactants including the mono- and diglyceride esters of fatty acids such as glyceryl monostearate, as used herein, are largely unregulated, and may be used according to good manufacturing practices. Ionic surfactants such as the anionic surfactant, sodium stearoyl lactylate (CAS Reg. No. 25-383-997) is typically limited to between approximately 0.2% and 0.5% of finished food products (see 21 CFR Section 172.846) For example, in milk or cream substitutes for coffee beverages, sodium stearoyl lactylate (SSL, as abbreviated herein) is limited to 0.3% by weight of the beverage. For an 8 oz serving, this translates to 0.72 g SSL. If the Myvatex P28 hybrid/mixed surfactant described above contains 50% by weight SSL, then as much as 1.4 g Myvatex P28 may be added to a serving of beverage. The SSL surfactant is approved for use in many other food products, including use in baked products, other dough products, coffee creamer, dehydrated potatoes, snack dips, cheese substitutes, sauces, gravies and any foods containing sauces or gravies, as well as in any prepared mixes for each of the above foods. Since sauces and mixes are very broad categories, and foods that may contain small amounts of sauces is even broader, SSL can properly be added to a wide variety if not limitless range of food products.

While SSL is a preferred anionic surfactant, other similar anionic surfactants may be substituted for SSL in the molecular hybrid anionic/nonionic binary surfactant system described. In many applications the surfactant should or must be approved by the FDA or other such applicable regulatory authority). For example, sodium stearyl fumarate may be combined with a nonionic surfactant. Similarly, other non-ionic surfactants may be substituted for glyceryl monostearate or glyceryl mono- and distearate, such as glyceryl monopalmitate, glyceryl monooleate and others.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the proportions of components used in the present compositions and to the manner in which the compositions are used. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention. Further, specification of a numerical range including values greater than one includes specific description of each integer value within that range.

Thus, additional embodiments are within the scope of the invention and within the following claims.

What is claimed is:

1. A water-dispersible dry-milled composition comprising
phytosterol-surfactant conglomerate (PSC) particles comprising
a blend of microparticulate phytosterols and a binary surfactant that comprises at least one non-ionic surfactant and at least one ionic surfactant; and
optionally further comprising one or more additional dry ingredients selected from the group consisting of anti-caking agents, anti-foam agents, natural and artificial sweeteners, non-dairy creamers, and flavoring agents.

2. The composition of claim 1
wherein said PSC particles comprise a blend of 1 part by weight of dry microparticulate non-ester phytosterols and from 0.2 to 2 parts by weight of a dry binary surfactant, wherein said binary surfactant comprises at least one non-ionic surfactant and at least one ionic surfactant combined in a weight ratio of between 0.3:1 and 3:1 respectively.

3. The composition of claim 1, wherein said composition is dispersible in hot and cold water.

4. The composition of claim 1 wherein said non-ionic surfactant is selected from the group consisting of monoglycerides and combinations of mono- and diglycerides.

5. The composition of claim 1 wherein said binary surfactant includes at least one non-ionic surfactant and at least one ionic surfactant combined in a weight ratio of between 0.5:1 and 2:1 respectively.

6. The composition of claim 1 wherein said binary surfactant includes: (i) mono- and diglycerides of stearic acid and (ii) sodium stearoyl lactylate combined in a weight ratio of between 0.3:1 and 3:1.

7. The composition of claim 1 comprising a milled blend of 1 part by weight of dry microparticulate phytosterols and between 0.3 and 1.5 parts by weight of a dry binary surfactant.

8. The composition of claim 1 wherein said phytosterols comprise non-esterified phytosterols.

9. The composition of claim 1 wherein said phytosterols comprise non-esterified phytostanols.

10. The composition of claim 1, wherein the average diameter of said PSC particles is 25-100 microns.

11. The composition of claim 1 wherein a premeasured amount of said composition is provided in the form of a powder or granules, and packaged in a single serving packet.

12. The composition of claim 1 wherein said composition is packaged and labeled as a consumer product for either food additive or dietary supplement use.

13. A dry, water-dispersible phytosterol composition, comprising
a composition resulting from the process of
dry milling a combination of dry particulate surfactant and a dry particulate phytosterol under high shear conditions.

14. The composition of claim 13, wherein said composition consists essentially of conglomerate particles comprising surfactant particles with associated phytosterol particles.

15. The composition of claim 13, wherein said surfactant is a binary surfactant.

16. A dry, water dispersible phytosterol composition, comprising
conglomerate particles, wherein said conglomerate particles comprise irregular binary surfactant particles associated together with phytosterol particles.

17. The composition of claim 16, wherein each conglomerate particle comprises at least one surfactant particle and at least one phytosterol particle.

18. A method for making a water dispersible phytosterol composition, comprising
blending and dry milling a mixture of dry particulate phytosterols and dry particulate binary surfactant under high shear conditions.

19. The method of claim 18, wherein said composition is hot and cold water dispersible.

20. The method of claim 18, wherein said dry blending produces phytosterol-surfactant conglomerate (PSC) particles.

21. A method for supplementing the diet of an individual with phytosterols, comprising
adding an amount of a dry, water dispersible composition comprising phytosterol-surfactant conglomerate (PSC) particles to a food or beverage item.

22. The method of claim 21, further comprising ingesting at least a portion of said food or beverage item.

23. The method of claim 21, wherein said amount is a cholesterol-lowering amount.
